(19) United States
(12) Patent Application Publication
(10) Pub. No.: US 2021/0145649 A1
(43) Pub. Date: May 20, 2021

(54) MEDICAL DEVICE FOR POSTOPERATIVE COMPRESSION FOLLOWING BREAST RECONSTRUCTION SURGERY

(71) Applicant: DECO MED SRL, Marcon (IT)

(72) Inventor: Giovanni BERTOLI, Marcon (IT)

(73) Assignee: DECO MED SRL, Marcon (IT)

(21) Appl. No.: 16/616,679

(22) PCT Filed: May 23, 2018
(86) PCT No.: PCT/IT2018/050089

§ 371 Date: Nov. 25, 2019

(30) Foreign Application Priority Data
May 24, 2017 (IT) 202017000056194

(51) Int. Cl. A61F 13/14; A41C 3/00; A41C 3/10

(52) U.S. Cl. CPC A61F 13/145; A61F 2013/0028; A41C 3/105; A41C 3/0064

(57) ABSTRACT

A device for postoperative compression of breast skin following breast reconstruction surgery includes a female garment that supports the breast, and wherein each cup of the garment contains inflatable modules with defined pressure gradients that generate different compressive orthogonal vectors on the four quadrants of the breast, leaving the areola-nipple complex free. The inflatable tubular modules have a shape resembling a truncated cone with variable thickness that is minimum in the area closest to the areola-nipple complex, and maximum in the area furthest away from the areola-nipple complex. The inflatable modules can be inflated separately. The device is made up of non-elastic tissue preventing the cups from moving away from breast skin when air chambers are inflated, and includes two non-elastic tissue straps that cross on the spine and close on the front. In the two sub-axillary areas, a Velcro clasp allows operation even when postoperative drainage is in place.

This application concerns a medical device for postoperative compression following breast reconstruction surgery after mastectomy for breast cancer.

Today breast surgery is performed for multiple purposes. Nipple sparing, skin sparing and skin reducing mastectomy, quadrantectomy and reconstruction with prostheses or flaps is the surgical treatment for cancer. For immediate reconstruction, it can make use of biological matrices that are used as “complementary fabric” to create the pocket where the prosthesis will be placed.

In non-immediate, two-time reconstructions, an expander is inserted under the greater pectoral muscle, and after it has performed its function, the expander is replaced by the final prosthesis during a second surgical procedure that is performed a few months later.

In aesthetic surgery an additive prosthesis is surgically inserted to increase breast volume. It can be inserted under the gland or under the muscle.

Mastopexy is a breast lifting procedure to control ptosis.

Several scientific studies have recently explored interactions between implanted biomaterials and patient tissues in order to understand the incorporation mechanisms and, especially, to limit postoperative complications, including, in the first place, serum production which, when uncontrolled, is the first step towards surgical failure.

A seroma is accumulation of fluid produced by the body as a result of the inflammatory process induced by surgery.

Within a range of 20-30 cc daily, it does not constitute a danger, but if daily fluid build-up is still around 100 cc 10 days after the implant, this is an alarm sign for the surgeon as it can constitute a risk for the onset of infection.

A seroma can still hinder intimate contact between the biological matrix, when implanted, and vascularised subcutaneous tissue because it is interposed between the two and, therefore, prevents the former's integration.

It can cause dehiscence of the surgical incision with subsequent exposure of the breast implant.

Well known scientific studies have proven that empty spaces inside the mammary chamber encourage serum build up during the postoperative period. Recent innovations in the field of surgical techniques encourage obliteration of dead spaces during surgery by suturing biological matrix and subcutaneous tissues, by using fibrin glue to make the various fasciae adhere together, and by using the biological matrix to form a taught lining for the breast prosthesis.

Even during the postoperative period, compression must be exerted on the breast for at least 15 days to ensure that subcutaneous tissues adhere to the implanted biomaterials to avoid the formation of dead spaces and, hence, serum build-up. The specific case of implantation of a submuscular expander presents the particular need to modulate the external compression gradient based on the variation in expansion of the expander itself, which shifts from 200 cc to ca. 400 cc in a medium breast over a period of six months. It is obvious that, as the internal submuscular expander is gradually inflated, external compression must be reduced to maintain a constant pressure gradient on breast skin.

Today, compression on breast skin is exerted during the postoperative phase through tissues that have a differentiated structure, which can be more or less compressed. These are the so-called containment sports bras or bandage bands, which feature a minimum or almost absent projection of the cup in order to exert frontal compression on the areola-nipple complex. These devices minimise breast movements but, apart from compression of the areola-nipple complex, they do not compress breast skin along an orthogonal axis.

Indeed, today there is no device that compresses certain areas of the breast in a targeted manner, applying less compression on other areas. Recent studies in the field of regenerative surgery and of the tissue repair process reveal how postoperative compression must be performed homogeneously on all breast skin, which is entirely involved in the inflammatory/regenerative process, especially if breast reconstruction was performed using biological matrices that, to become self-tissue, need intimate contact with subcutaneous tissue.

Even during the postoperative phase, this area needs compression to maintain said adherence for at least 15 days.

The only point that can be maintained free of pressure gradients is the areola-nipple complex, since this part of the breast is more susceptible to perfusion problems and, therefore, to a greater risk of cutaneous necrosis.

Frontal compression of the areola-nipple complex is not recommended, but orthogonal compression to the skin surface must be exerted on the four quadrants of the breast to ensure stable intimate contact between subcutaneous tissue and prosthetic implants in order to obliterate dead spaces where serum can build-up inside mammary spaces.

Today certain devices and intimate clothing items possess inflatable structures. But these structures are completely different and are designed for other purposes.

For instance, U.S. Pat. No. 9,468,236 claims an intimate garment, a bra (and not a medical device like this invention) with an inflatable structure in each cup, which is more or less spherical, is not divided into quadrants, has no varied pressure gradients, and is positioned in the inferior area of the cup for the sole purpose of making the bra more comfortable. The device is studied to better adapt to the woman's body without absolutely solving the problem of pressure gradient required to obliterate dead spaces.

### SCOPE

The main purpose of this invention is, therefore, to solve the technical issues underscored by eliminating the inconveniences of currently available medical devices.

In fact, the purpose of this invention is to provide compressive and variable orthogonal vectors along the four quadrants of the breast, leaving the central area that corresponds to the areola-nipple complex free. This bra has minimally projecting cups, which internally contain, in the area that interfaces with the skin of the operated breast, several air chambers designed to be inflated with precise mmHg values in order to exert adjustable orthogonal compression on breast skin.

This device ensures obliteration of dead spaces inside the breast chamber between the various implanted materials and vital tissues.

Obliteration of dead spaces diminishes the risk of serum build-up, favouring excellent postoperative recovery.

The invention presents as a bra with two non-preformed cups that are totally closed for 360° without a cleavage between the breasts, and which contain modules that can be inflated according to defined pressure gradients. It is made of non-elastic tissue particularly in the anterior area under the breast groove, and in the subaxillary and posterior area. It also includes two straps that cross at the back and close like a belt on the front.

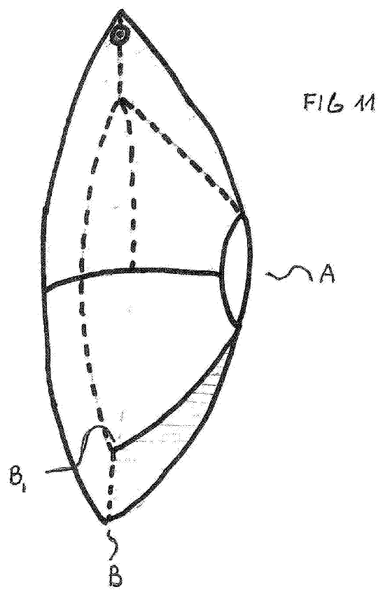

The different pressure gradients are created by introducing various quantities of air, and also by the shape of the inflatable modules. A sagittal section (FIG. 9-10-11) reveals that they are tapered near the areola-nipple complex and are broader towards the chest. Hence lesser pressure can be exerted on the area adjacent to the areola-nipple complex, with enhanced pressure on peripheral breast skin.

The non-elastic tissue prevents the cups from moving away from breast skin when the air chambers are inflated. Hence it ensures that the cups adhere to the breast, allowing air chamber expansion to perform the established axial compression on the breast skin with which it interfaces.

The above straps are made of non-elastic tissue (FIG. 1-2-3-4-5-6-7-8). A Velcro clasp in the subaxillary area allows the bra to be used even with drainage tubes (FIG. 3). The overall item is a band that allows thoracic expansion for breathing but which, at the same time, maintains compression on breast quadrants.

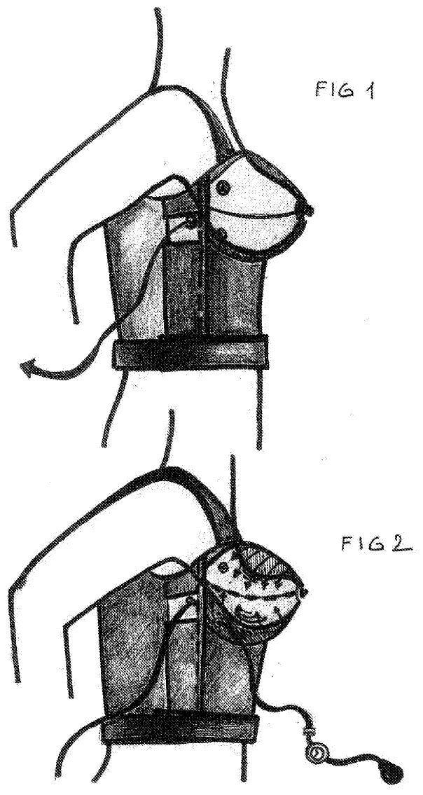

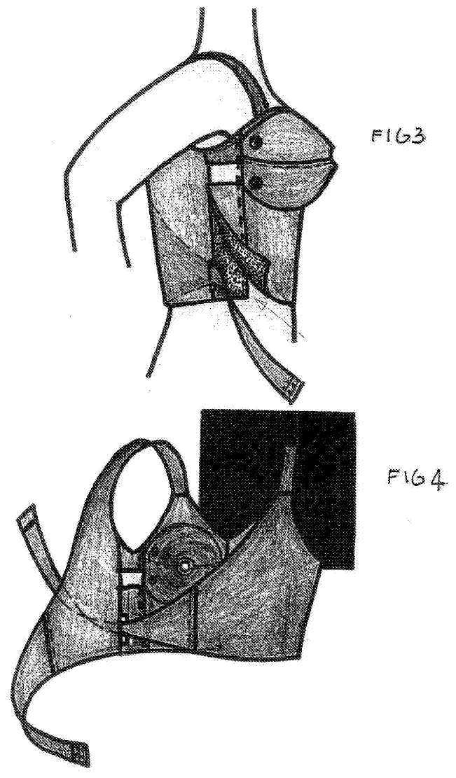

The hollow part of every cup that is in contact with the skin has air chambers in a number ranging from one to a multiple of one, placed in a configuration to spare the areola-nipple complex from pressure, and to involve it when clinically required (e.g., mastopexy or quadrantectomy) (FIG. 1-2).

Air chamber pressure values are defined by the surgeon, depending on the need for compression, and are applied on the various quadrants of the breast, depending on clinical needs, by means of air injected through a manual pump that, along with the pressure gauge, is fitted, from time to time, to the various non-allergic plastic valves in the various quadrants of the breast (FIG. 1-2-3).

As with breast skin, when operated to remove lymph nodes, the armpit too could need compression designed to obliterate empty spaces by means of bringing internal tissues together. The invention, second element of the system, is shaped according to the complex anatomy of shoulder and armpit.

It is made up of an element, a broad and fitting shoulder strap (FIG. 4-5-6), that is connected through an extension of non-elastic tissue towards the armpit, to the underarm that internally contains, in contact with the skin, an inflatable air chamber that exerts an orthogonal pressure gradient on the axillary cavity. This element can be connected to the compressive bra by means of LYCRA Fabric® or VELCRO® to prevent it from slipping laterally.

Additional characteristics and benefits of the invention will be underscored by a detailed description of a specific feature, a non-exclusive form of construction that is illustrated as a rough indication and is not limited to the enclosed drawings in which:

FIG. 1 section of the invention with the air chamber not inflated;

FIG. 2 section of the invention with inflated air chambers;

FIG. 3 lateral view of the invention without tissue lining;

FIG. 4 posterior view of the invention without tissue lining;

FIG. 5 open posterior view of the invention without tissue lining;

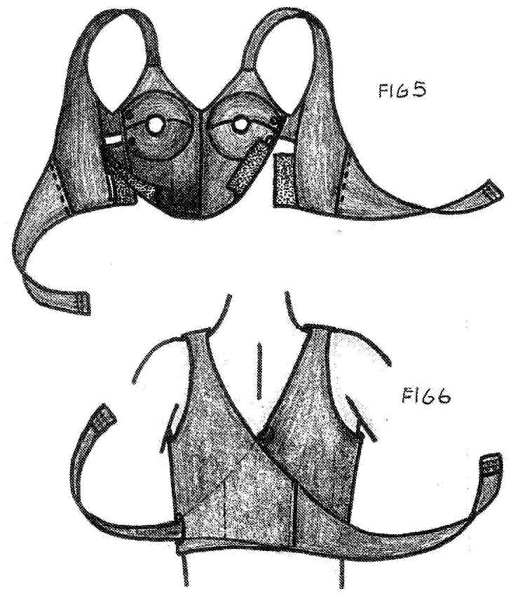

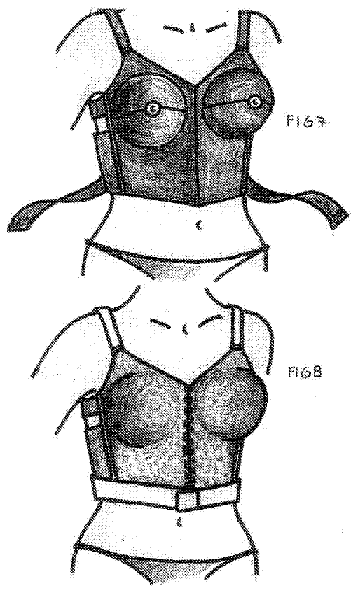

FIG. 6 posterior view of the invention, clasped, with tissue lining;

FIG. 7 front view of the invention, without tissue lining;

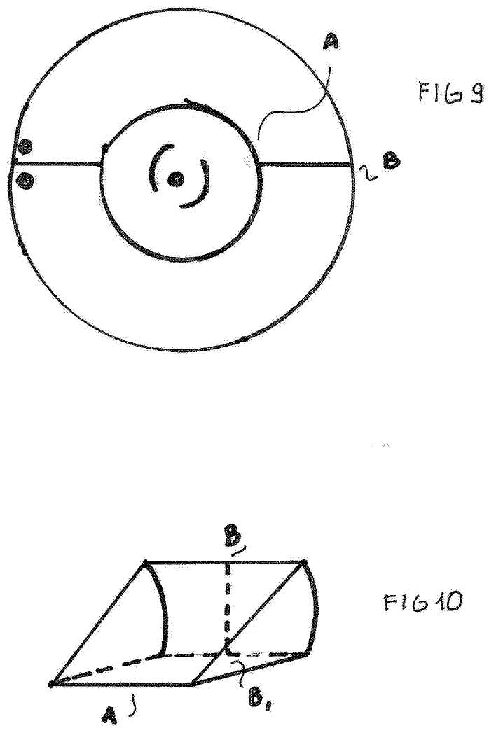

FIG. 8 front view of the invention without tissue lining;

FIG. 9-10-11 illustrates the inflatable modules (air chambers), front view and section. In A the air chamber has a height (thickness) of zero, which is constant along the entire circumference. This height (thickness) increases proportionately as B draws close, and the air chamber has a constant height (thickness) B-B1 along the entire circumference.

It must be said that all information already known during the patent application procedure is intended as not claimed and the subject of disclaimer. In any case, the materials used and the dimensions of the invention can be more relevant depending on the specific needs.

The various means to perform certain different functions must not coexist only in the form of product described, but can be present in many forms of product that are even not illustrated.

The characteristics indicated as benefits, appropriate and so one can also be removed.

1. A medical device for the postoperative compression of the skin after breast reconstruction, comprising a female breast support garment, wherein each cup of the garment comprises inflatable modules that, according to defined, pressurized gradients, achieve orthogonal, compressive and different vectors on the four quadrants of the breast, leaving empty the areolar nipple complex.

2. The medical device as in claim 1, whose inflatable tubular modules have, together, a defined shape similar to a trunked cone with variable thickness.

3. The medical device as claimed in claim 1, whose inflatable tubular modules have a minimum thickness (equal to zero) at the nearest part of the areolar nipple complex and maximum at the far end of the areolar nipple complex.

4. The medical device as claimed in claim 1, wherein the inflatable modules are 2 or multiple of 2 and are inflatable separately through the valves.

5. The medical device as claimed in claim 1, wherein it is constituted by inelastic material which has antagonistic function from the removal from the skin of the cups, exerted by inflating the air chambers.

6. The medical device as claimed in claim 1, comprising two inelastic straps that intersect on the back and then close like a waistband in the front.

7. The medical device as claimed in claim 1, wherein there is a closure with velcro in the two sub underarm parts so as to allow insertion of the invention even after operative drainage.
